(19) United States
(12) Patent Application Publication
(10) Pub. No.: US 2022/0184396 A1
(43) Pub. Date: Jun. 16, 2022

(54) RAPID NEURAL RESPONSE TELEMETRY CIRCUIT AND SYSTEM OF COCHLEAR IMPLANT

(71) Applicant: ZHEJIANG NUROTRON BIOTECHNOLOGY CO., LTD., HANGZHOU, Zhejiang Province (CN)

(72) Inventors: SUI HUANG, HANGZHOU, ZHEJIANG PROVINCE (CN); LIAN NI, HANGZHOU, ZHEJIANG PROVINCE (CN); XIAOAN SUN, HANGZHOU, ZHEJIANG PROVINCE (CN)

(21) Appl. No.: 17/439,779

(22) PCT Filed: Jul. 19, 2019
(86) PCT No.: PCT/CN2019/096646

§ 371 Date: Sep. 15, 2021

(30) Foreign Application Priority Data
Mar. 15, 2019 (CN) 201910197221.5

(51) Int. Cl. A61N 1/36; A61N 1/05; H04R 25/00

(52) U.S. Cl. CPC A61N 1/36038; A61N 1/0541; A61N 1/36034; H04R 25/505; H04R 25/606

(57) ABSTRACT

The present invention discloses a rapid neural response telemetry (NRT) circuit and system of a cochlear implant. The circuit comprises a stimulus generator, a signal amplifier, an analog-to-digital (A/D) converter and a calculated data memory. The stimulus generator zero charges in a nerve tissue before stimulus onset and offset, and the onset asynchrony of two continuous stimuli on the same electrode may be adjusted at will. The signal amplifier filters and amplifies nervous impulse signals that are evoked by electric stimuli and received by a collector electrode. The A/D converter can adjust the sampling frequency and start-up time, and is connected to the signal amplifier to perform A/D conversion on amplified analog signals. The calculated data memory is connected to the A/D converter to calculate and store the data undergoing A/D conversion. According to the present invention, a stimulus circuit is improved to reduce artifacts of NRT so that key parameters for NRT can be flexibly controlled, the success rate in eliciting NRT is improved, and the NRT speed is greatly improved by calculating and storing the data.

### TECHNICAL FIELD OF THE INVENTION

The present invention belongs to the field of implantable medical devices, and particularly relates to a rapid neural response telemetry (NRT) circuit and system of a cochlear implant.

### BACKGROUND OF THE INVENTION

The neural response telemetry (NRT) technology refers to the use of the internal circuit of the cochlear implant on the designated non-stimulating electrode to collect the electrical stimulation induced potential formed by the stimulation of the implant after the designated electrode is stimulated. As it requires no other auxiliary equipment and has the advantages of direct results and ease of use, NRT is now an important reference for physicians during surgery to determine the success of implantation and for the mapping process for infants who do not have the ability to provide subjective feedback.

In actual practice, weak neural response signals are easily interfered by artificial electric stimulus artifacts and other noises, so it is very difficult to collect accurate neural response signals. Forward masking subtraction method is currently the most commonly used method in cochlear implant NRT, which elicits (A) probe stimulus, (B) masking stimulus+probe stimulus, (C) masking stimulus, and (D) no stimulus based on the principle that the nerve will not respond to any other electric stimuli for a period of time after stimulus onset, and calculates data from the four cases according to a rule of A-B+C-D and then averages the calculated data several times to obtain a final neural response waveform. This algorithm is too demanding on a neural response telemetry circuit of the cochlear implant which is required to be able to flexibly control the onset asynchrony of masking stimulus and probe stimulus, the offset cancellation time of an amplifier circuit, and the sampling frequency and start-up time delay of an analog-to-digital (A/D) conversion circuit. Moreover, this algorithm has a major disadvantage of a relatively slow speed especially in case that mass two-way communication is required between a PC (personal computer) terminal and the implant, which causes inconvenience to physicians during surgery and to the mapping process for infants.

### SUMMARY OF THE INVENTION

In view of this, an object of the present invention is to provide a rapid neural response telemetry (NRT) circuit and system of a cochlear implant. The circuit can reduce the interference of stimulus artifacts on neural response, improve the success rate in eliciting NRT by flexibly controlling the interval between two stimuli, the interval for zeroing DC charges between electrodes, the offset cancellation time of an amplifier, and the sampling frequency and start-up delay time of an analog-to-digital (A/D) converter, and significantly improve the NRT speed by adding and subtracting A/D conversion signals according to a certain rule, storing the data after addition and subtraction, and finally sending the data to a mapping device of speech processor in one go.

To achieve the object, the present invention provides a rapid neural response telemetry circuit of a cochlear implant, at least comprising a stimulus generator, a signal amplifier, an A/D converter and a calculated data memory, wherein

the stimulus generator comprises a stimulus control module, a stimulus control timer, a switch S1, a switch S2 and an AC stimulus module, wherein

the stimulus control module is connected to the AC stimulus module and the switches S1 and S2 to generate AC stimulus current between a stimulus electrode and a return electrode of the AC stimulus module through digital signal control and to zero charges at both ends after stimulus offset;

the stimulus control timer is connected to the stimulus control module to record the time the onset asynchrony of two continuous stimuli generated by the stimulus control module on the same electrode;

the switch S1 is connected to the stimulus electrode, the switch S2 is connected to the return electrode, and the switches S1 and S2 are turned on and simultaneously connected to a fixed level before stimulus onset and after stimulus offset;

the AC stimulus module generates AC stimulus current between the stimulus electrode and the return electrode, and the magnitude and pulse width of the stimulus current are controlled by the stimulus control module;

the signal amplifier comprises a low-pass filtering (LPF) module, an offset cancellation amplifier module and an offset cancellation timer, wherein

the LPF module is connected to the stimulus electrode and the return electrode to filter high-frequency noises of received tiny nervous impulse signals;

the offset cancellation amplifier module is connected to the LPF module to amplify output signals of the LPF module, and cancels its own offset signals;

the offset cancellation timer is connected to the offset cancellation amplifier module to control the offset cancellation time;

the A/D converter comprises an analog-to-digital conversion (ADC) circuit, a frequency dividing circuit and a start-up timer, wherein

the ADC circuit is connected to the offset cancellation amplifier module to perform A/D conversion on amplified signals;

the frequency dividing circuit is connected to the ADC circuit to control the sampling frequency of the ADC circuit;

the start-up timer is connected to the ADC circuit to control the start-up time of the ADC circuit;

the calculated data memory comprises a primary data register, a calculator and a calculated data register, wherein

the primary data register is connected to the ADC circuit to store the data generated by the ADC circuit; and

the calculator is connected to the primary data register and the calculated data register to add and subtract data in the primary data register and the calculated data register based on a cochlear implant NRT algorithm and to store calculated results in the calculated data register.

Preferably, the switches S1 and S2 are automatically turned off before stimulus onset and automatically turned on after stimulus offset, so as to remove stimulus artifacts and residual DC charges between electrodes.

Preferably, the range of the stimulus control timer is 100 μs to 1000 μs.

Preferably, the sampling frequency of the ADC circuit may vary from 10K to 10 MHz.

Preferably, the start-up time of the ADC circuit is within the range of 0 μs to 500 μs.

Preferably, the measurement accuracy of the ADC circuit is 6 bits to 18 bits.

To achieve the above object, the present invention further comprises a rapid neural response telemetry system of a cochlear implant, further comprising PC application software, a forward transmission module, a command decoding module, a reverse transmission module and a reverse demodulation module, wherein

the PC application software is connected to the forward transmission module and the reverse demodulation module to send NRT command parameters to the rapid neural response telemetry circuit of the cochlear implant through the forward transmission module and/or graphically display data sent back from the reverse modulation module, so that users can obtain clear neural response waveforms;

the forward transmission module is connected to the command decoding module in a wireless transmission mode to encode, modulate and transmit NRT parameters configured by the PC application software;

the command decoding module is connected to the rapid neural response telemetry circuit of the cochlear implant to control the stimulus control module, the stimulus control timer, the offset cancellation timer, the start-up timer, the frequency dividing circuit and the calculator;

the reverse transmission module is connected to the calculated data register to modulate the data in the calculated data register and reversely transmit the data out of body; and

the reverse demodulation module is connected to the reverse transmission module in a wireless induction mode to demodulate and digitize the data transmitted from the reverse transmission module and then to transmit the data to the PC application software.

The beneficial effect of the present invention is that the circuit can reduce the interference of stimulus artifacts on neural response by improving the circuit of the stimulus generator, improve the success rate in eliciting NRT by flexibly controlling the onset asynchrony of two stimuli, the offset cancellation time of the amplifier, and the sampling frequency and start-up time of the A/D converter, and significantly improve the NRT speed by adding and subtracting A/D conversion signals according to a certain rule, storing the data after addition and subtraction, and finally sending the data to the mapping device of speech processor in one go.

### BRIEF DESCRIPTION OF THE DRAWINGS

In order to make the object, technical solutions and beneficial effects of the present invention clearer, the present invention will be described with reference to following accompanying drawings.

FIG. 1 is an overall block diagram of a specific application example of a rapid neural response telemetry circuit of a cochlear implant according to an embodiment of the present invention;

FIG. 2 is a specific block diagram of a specific application example of a rapid neural response telemetry system of a cochlear implant according to an embodiment of the present invention;

FIG. 3 is a schematic diagram of a forward masking subtraction method of a specific application example of the rapid neural response telemetry circuit of a cochlear implant according to the embodiment of the present invention;

FIG. 4 is a neural response signal diagram at different stimulus onset asynchronies of a specific application example of the rapid neural response telemetry system of a cochlear implant according to the embodiment of the present invention; and

FIG. 5(a), FIG. 5(b) and FIG. 5(c) are a comparison of control waveforms for the ADC circuit start-up time and the interval for zeroing DC charges of the rapid neural response telemetry system of a cochlear implant according to the embodiment of the present invention.

### DETAILED DESCRIPTION OF THE INVENTION

Preferred embodiments of the present invention will be described in detail below with reference to the accompanying drawings.

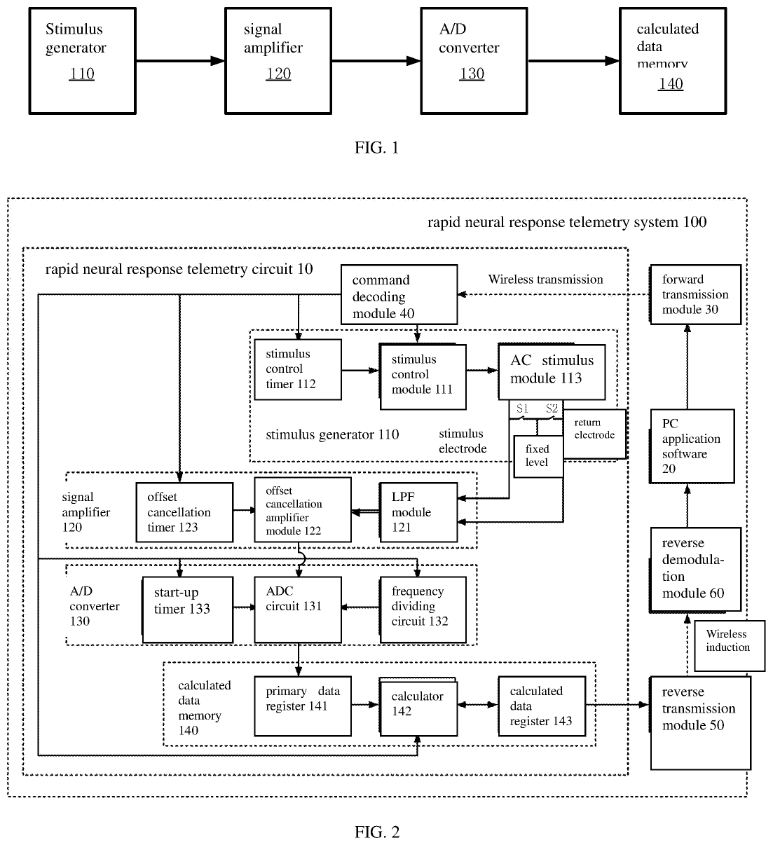

Referring to FIGS. 1 to 2, an overall block diagram of a rapid neural response telemetry circuit 10 of a cochlear implant and a specific block diagram of a rapid neural response telemetry system 100 of a cochlear implant according to an embodiment of the present invention are shown.

A rapid neural response telemetry circuit 10 of a cochlear implant is provided, at least comprising a stimulus generator 110, a signal amplifier 120, an A/D converter 130 and a calculated data memory 140.

The stimulus generator 110 comprises a stimulus control module 111, a stimulus control timer 112, a first switch S1, a second switch S2 and an AC stimulus module 113.

The stimulus control module 111 is connected to the AC stimulus module 112 and the first and second switches S1 and S2 to generate AC stimulus current between a stimulus electrode and a return electrode of the AC stimulus module 112 through digital signal control and to zero charges at both ends after stimulus offset.

The stimulus control timer 113 is connected to the stimulus control module 111 to time the onset asynchrony of two continuous stimuli generated by the stimulus control module 111 on the same electrode.

The first switch S1 is connected to the stimulus electrode, the second switch S2 is connected to the return electrode, and the first and second switches S1 and S2 are turned on and simultaneously connected to a fixed level before stimulus onset and after stimulus offset.

The AC stimulus module 112 generates AC stimulus current between the stimulus electrode and the return electrode, and the magnitude and pulse width of the stimulus current are controlled by the stimulus control module 111.

The signal amplifier 120 comprises a low-pass filtering (LPF) module 121, an offset cancellation amplifier module 122 and an offset cancellation timer 123.

The LPF module 121 is connected to the stimulus electrode and the return electrode to filter high-frequency noises of received tiny nervous impulse signals.

The offset cancellation amplifier module 122 is connected to the LPF module 121 to amplify output signals of the LPF module 121 and may cancel its own offset signals.

The offset cancellation timer 123 is connected to the offset cancellation amplifier module 122 to control the offset cancellation time.

The A/D converter 130 comprises an analog-to-digital conversion (ADC) circuit 131, a frequency dividing circuit 132 and a start-up timer 133.

The ADC circuit 131 is connected to the offset cancellation amplifier module 122 to perform A/D conversion on amplified signals.

The frequency dividing circuit 132 is connected to the ADC circuit 131 to control the sampling frequency of the ADC circuit.

The start-up timer 133 is connected to the ADC circuit 131 to control the start-up time of the ADC circuit.

The calculated data memory 140 comprises a primary data register 141, a calculator 142 and a calculated data register 143.

The primary data register 141 is connected to the ADC circuit 131 to store the data generated by the ADC circuit 131.

The calculator 142 is connected to the primary data register 141 and the calculated data register 143 to add and subtract data in the primary data register 141 and the calculated data register 143 based on a cochlear implant NRT algorithm and to store calculated results in the calculated data register 143.

To achieve the above object, referring to FIG. 2, the present invention further comprises a rapid neural response telemetry system 100 of a cochlear implant, further comprising PC application software 20, a forward transmission module 30, a command decoding module 40, a reverse transmission module 50 and a reverse demodulation module 60.

The PC application software 20 is connected to the forward transmission module 30 and the reverse demodulation module 60, and may send NRT command parameters to the rapid neural response telemetry circuit 10 of the cochlear implant through the forward transmission module 30 and may also graphically display data sent back from the reverse modulation module 60, so that users can obtain clear neural response waveforms.

The forward transmission module 30 is connected to the command decoding module 40 in a wireless transmission mode to encode, modulate and transmit NRT parameters configured by the PC application software 20.

The command decoding module 40 is connected to the rapid neural response telemetry circuit 10 of the cochlear implant to control the stimulus control module 111, the stimulus control timer 113, the offset cancellation timer 123, the start-up timer 133, the frequency dividing circuit 132 and the calculator 142.

The reverse transmission module 50 is connected to the calculated data register 143 to modulate the data in the calculated data register 143 and reversely transmit the data out of body.

The reverse demodulation module 60 is connected to the reverse transmission module 50 in a wireless induction mode to demodulate and digitize the data transmitted from the reverse transmission module 50, and transmit the data to the PC application software 20.

Further, according to the rapid neural response telemetry circuit of the cochlear implant, the switches S1 and S2 are automatically turned off before stimulus onset and automatically turned on after stimulus offset, so as to remove stimulus artifacts and residual DC charges between electrodes.

Further, according to the rapid neural response telemetry circuit of the cochlear implant, the range of the stimulus control timer 113 is 100 μs to 1000 μs.

Further, according to the rapid neural response telemetry circuit of the cochlear implant, the sampling frequency of the ADC circuit 131 may vary from 10K to 10 MHz.

Further, according to the rapid neural response telemetry circuit of the cochlear implant, the start-up time of the ADC circuit 131 is within the range of 0 μs to 500 μs.

Further, according to the rapid neural response telemetry circuit of the cochlear implant, the measurement accuracy of the ADC circuit 131 is 6 bits to 18 bits.

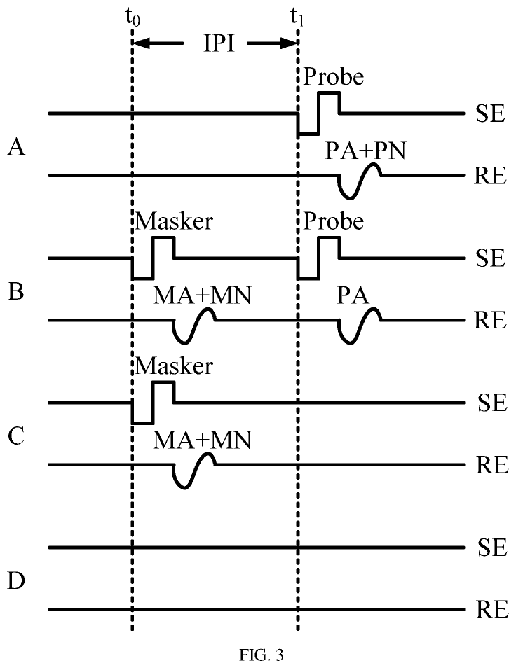

FIG. 3 is a schematic diagram of a forward masking subtraction method of a specific application example of the rapid neural response telemetry circuit 10 of a cochlear implant according to the embodiment of the present invention. In the figure, SE represents a stimulus waveform of the stimulus electrode, RE represents a waveform received by a receiver electrode, “Probe” represents a probe stimulus waveform, “Masker” represents a masking stimulus waveform, PA represents an artifact waveform caused by the probe stimulus waveform, PN represents a neural response waveform caused by the probe stimulus, MA represents an artifact caused by the masking stimulus waveform, and MN represents a neural response waveform caused by the masking stimulus. In case A, the cochlear implant has one probe stimulus. In case B, the cochlear implant has one masking stimulus and one probe stimulus, with the onset time of the probe stimulus the same as that in case A. In case C, the cochlear implant has one masking stimulus, with the onset time of the masking stimulus the same as that in case B. In case D, the cochlear implant has no stimulus. By receiving the signals in the four cases respectively, the data are calculated according to a rule of A-B+C-D and averaged several times based on the principle that the nerve after onset of two fast continuous stimuli will not respond to the second stimulus, and finally the influence of PA, MA and system background noise on the cochlear implant NRT can be eliminated. The calculator 142 adds and subtracts the data in the primary data register 141 and the calculated data register 143 based on the forward masking subtraction algorithm, stores the results in the calculated data register 143, and deduces a calculated result once after several calculations, thus greatly improving the NRT speed.

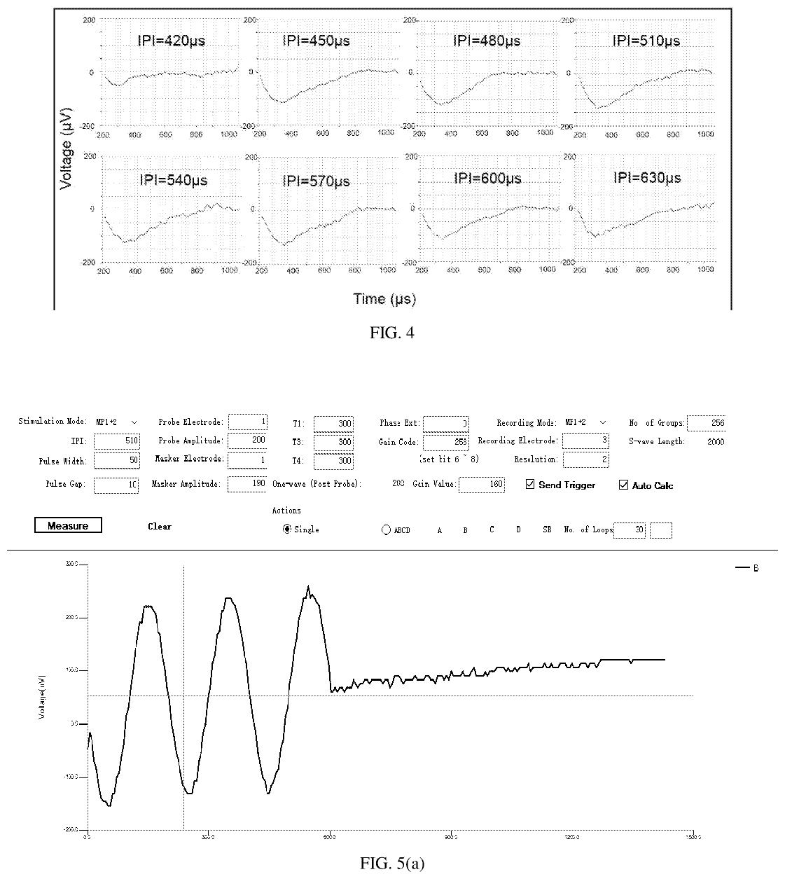

FIG. 4 is a neural response signal diagram at different stimulus onset asynchronies of a specific application example of the rapid neural response telemetry system of a cochlear implant according to the embodiment of the present invention. Because the neural response time and the anergy time to the second stimulus vary among individuals, it is necessary to flexibly adjust the inter-pulse interval (IPI) in the NRT practice. FIG. 4 shows different NRT signals received at different IPIs from 420 μs to 630 μs using the rapid neural response telemetry system 100 of the cochlear implant, where the X-coordinate is the time (100 μs per division) and the Y-coordinate is the voltage (50 microvolts per division). The comparison in the figure shows that the NRT signals measured at different IPIs vary. When a patient has an IPI of 510 microseconds, the amplitude of the neural response signal reaches the maximum.

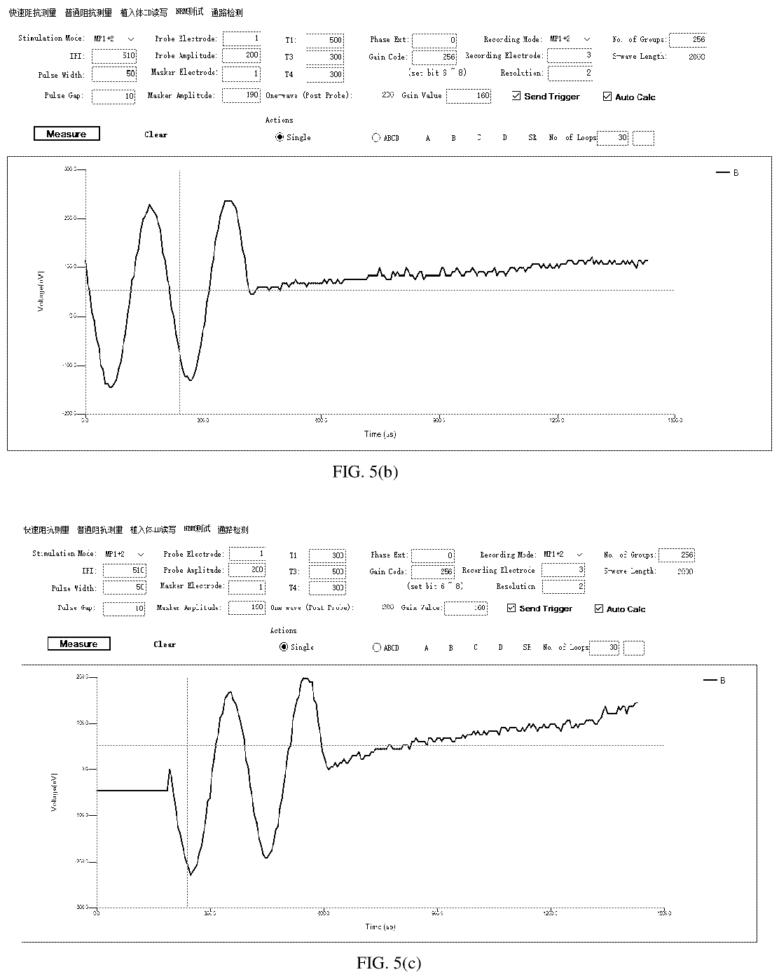

FIG. 5 is a comparison of control waveforms for the ADC circuit start-up time and the interval for zeroing DC charges of the rapid neural response telemetry system of a cochlear implant according to the embodiment of the present invention, where the X-coordinate is the time (300 μs per division) and the Y-coordinate is the voltage (100 microvolts per division). FIG. 5(a) is a small signal waveform obtained under normal conditions, and FIG. 5(b) is a small signal waveform obtained by delaying the start-up time of the ADC circuit by 200 m. Compared with FIG. 5(a), it can be seen that the waveform is truncated by 200 m. FIG. 5(c) is a small signal waveform obtained by extending the DC charge zeroing time by 200 m. Compared with FIG. 5(a), since the stimulus electrode is connected to the return electrode in the first 200 m, the waveform of the received small signals is a flat line.

According to the present invention, the circuit can reduce the interference of stimulus artifacts on neural response, improve the success rate in eliciting NRT by flexibly controlling the onset asynchrony of two stimuli, the interval for zeroing DC charges between electrodes, the offset cancellation time of the amplifier, and the sampling frequency and start-up time of the A/D converter, and significantly improve the NRT speed by adding and subtracting A/D conversion signals according to a certain rule, storing the data after addition and subtraction, and finally sending the data to a mapping is device of speech processor in one go. Moreover, the entire circuit has the advantages of strong adaptability and easy integration.

Finally, it should be noted that the above preferred embodiments are not used for limiting but merely for describing the technical solutions of the present invention. Although the present invention has been described in detail by the above preferred embodiments, it should be understood by those of skill in the art that various modifications can be made thereto in form and detail without deviating from the scope defined by the claims of the present invention.

1. A rapid neural response telemetry circuit of a cochlear implant, at least comprising a stimulus generator, a signal amplifier, an analog-to-digital (A/D) converter and a calculated data memory, wherein
the stimulus generator comprises a stimulus control module, a stimulus control timer, a first switch S1, a second switch S2 and an AC stimulus module, wherein
the stimulus control module is connected to the AC stimulus module and the first and second switches S1 and S2 to generate AC stimulus current between a stimulus electrode and a return electrode of the AC stimulus module through digital signal control and to zero charges at both ends after stimulus offset;
the stimulus control timer is connected to the stimulus control module to time the onset asynchrony of two continuous stimuli generated by the stimulus control module on the same electrode;
the first switch S1 is connected to the stimulus electrode, the second switch S2 is connected to the return electrode, and the first and second switches S1 and S2 are turned on and simultaneously connected to a fixed level before stimulus onset and after stimulus offset;
the AC stimulus module generates AC stimulus current between the stimulus electrode and the return electrode, and the magnitude and pulse width of the stimulus current are controlled by the stimulus control module;
the signal amplifier comprises a low-pass filtering (LPF) module, an offset cancellation amplifier module and an offset cancellation timer, wherein
the LPF module is connected to the stimulus electrode and the return electrode to filter high-frequency noises of received tiny nervous impulse signals;
the offset cancellation amplifier module is connected to the LPF module to amplify output signals of the LPF module, and cancels its own offset signals;
the offset cancellation timer is connected to the offset cancellation amplifier module to control the offset cancellation time;
the A/D converter comprises an analog-to-digital conversion (ADC) circuit, a frequency dividing circuit and a start-up timer, wherein
the ADC circuit is connected to the offset cancellation amplifier module to perform A/D conversion on amplified signals;
the frequency dividing circuit is connected to the ADC circuit to control the sampling frequency of the ADC circuit;
the start-up timer is connected to the ADC circuit to control the start-up time of the ADC circuit;
the calculated data memory comprises a primary data register, a calculator and a calculated data register, wherein
the primary data register is connected to the ADC circuit to store the data generated by the ADC circuit; and
the calculator is connected to the primary data register and the calculated data register to add and subtract data in the primary data register and the calculated data register based on a cochlear implant NRT algorithm and to store calculated results in the calculated data register.

2. The rapid neural response telemetry circuit of a cochlear implant according to claim 1, wherein the first and second switches S1 and S2 are automatically turned off before stimulus onset and automatically turned on after stimulus offset, so as to remove stimulus artifacts and residual DC charges between electrodes.

3. The rapid neural response telemetry circuit of a cochlear implant according to claim 1, wherein the range of the stimulus control timer is 100 μs to 1000 μs.

4. The rapid neural response telemetry circuit of a cochlear implant according to claim 1, wherein the sampling frequency of the ADC circuit may vary from 10K to 10 MHz.

5. The rapid neural response telemetry circuit of a cochlear implant according to claim 1, wherein the start-up time of the ADC circuit is within the range of 0 m to 500 m.

6. The rapid neural response telemetry circuit of a cochlear implant according to claim 1, wherein the measurement accuracy of the ADC circuit is 6 bits to 18 bits.

7. A system adopting the rapid neural response telemetry circuit of a cochlear implant according to claim 1, further comprising PC application software, a forward transmission module, a command decoding module, a reverse transmission module and a reverse demodulation module, wherein
the PC application software is connected to the forward transmission module and the reverse demodulation module to send NRT command parameters to the rapid neural response telemetry circuit of the cochlear implant through the forward transmission module and/or graphically display data sent back from the reverse modulation module, so that users can obtain clear neural response waveforms;
the forward transmission module is connected to the command decoding module in a wireless transmission mode to encode, modulate and transmit NRT parameters configured by the PC application software;
the command decoding module is connected to the rapid neural response telemetry circuit of the cochlear implant to control the stimulus control module, the stimulus control timer, the offset cancellation timer, the start-up timer, the frequency dividing circuit and the calculator;
the reverse transmission module is connected to the calculated data register to modulate the data in the calculated data register and reversely transmit the data out of body; and
the reverse demodulation module is connected to the reverse transmission module in a wireless induction mode to demodulate and digitize the data transmitted from the reverse transmission module and then to transmit the data to the PC application software.
